(19) United States
(12) Patent Application Publication
(10) Pub. No.: US 2015/0299761 A1
(43) Pub. Date: Oct. 22, 2015

(54) Substrates and Methods for Preparing Samples for Mass Spectrometry

(71) Applicant: Virgin Instruments Corporation, Sudbury, MA (US)

(72) Inventor: Stephen J. Hattan, Lowell, MA (US)

(73) Assignee: VIRGIN INSTRUMENTS CORPORATION, Sudbury, MA (US)

(21) Appl. No.: 14/690,399

(22) Filed: Apr. 18, 2015

Related U.S. Application Data
(60) Provisional application No. 61/982,248, filed on Apr. 21, 2014.

(51) Int. Cl. C12Q 1/37; G01N 1/40; G01N 33/68; H01J 49/02; H01J 49/16

(52) U.S. Cl. CPC C12Q 1/37; H01J 49/02; H01J 49/164; G01N 33/6851; G01N 1/4044

(57) ABSTRACT

A mass spectrometry substrate for performing adsorption and biological processing includes a porous silica-based material comprising a chemical adsorption material that captures analyte(s) of interest. A biologically active material incorporated into the porous silica-based material performs biological processing on the analyte(s) of interest.

### CROSS-REFERENCE TO RELATED APPLICATION

This application is a non-provisional application of U.S. Provisional Patent Application Ser. No. 61/982,248 filed Apr. 21, 2014, entitled “Substrates and Methods for Preparing Samples for Mass Spectrometry.” The entire disclosure of U.S. Provisional Patent Application Ser. No. 61/982,248 is incorporated herein by reference.

The section headings used herein are for organizational purposes only and should not to be construed as limiting the subject matter described in the present application in any way.

### INTRODUCTION

Studies of biological materials and the operations of biological systems are of interest to numerous scientific disciplines, and much investigation has focused on the appropriate means of procuring, processing, detecting and quantifying biological samples. These investigations are carried out in hopes of gaining insight into the functions and manner by which the various components of complex biological systems operate under a given set of natural or imposed conditions. Critical to most studies of complex biological systems is a means of separating the various components in such a way as to make them more amenable to detection and quantification. Separation of the materials under study is often necessary due to their diverse numbers, physical characteristics, chemical characteristics and large dynamic range in concentration. Thus, some components are present at much higher concentration levels than others. Separation stratagems of chromatography and gel electrophoresis are often used for this purpose, and are capable of being adapted to operate via different modes to take advantage of the different physical or chemical properties of proteins and peptides (e.g. size, charge, hydrophobicity, etc.), in order to achieve separation.

### BRIEF DESCRIPTION OF THE DRAWINGS

FIG. 1 illustrates an embodiment of porous glass fiber and porous membranes and discs according to the present teaching.

FIG. 2 illustrates an embodiment of sintered glass frits according to the present teaching.

FIG. 3 illustrates an embodiment of porous glass plates according to the present teaching.

FIG. 4A illustrates a chemical diagram for 3-glycidoxypropyl trimethoxysilane, a commonly available silane-compound that is used for surface modification according to the present teaching.

FIG. 4B illustrates a chemical diagram for n-octadecyltrimethoxysilane, a commonly available silane-compound that is used for surface modification according to the present teaching.

FIG. 5 illustrates a reaction mechanism wherein the 3 methoxy pendants on both molecules in FIGS. 4A and 4B may be induced to react with one another, as well as with hydroxyl functionalities present on silica surfaces, to form a stable covalent bond to the surface of the glass for surface modification.

FIG. 6 illustrates LC chromatograms of a Bovine Serum Albumin after the addition of 5 mm² piece of capture/digestion membrane according to the present teaching.

FIG. 7A illustrates a schematic of an electro-blotting procedure according to the present teaching.

FIG. 7B illustrates a resulting post blot membrane that can be cut to preserve separation.

FIG. 7C illustrates a mass spectrum of slices that are eluted to release peptides for analysis or further processing.

FIG. 8A illustrates a plot of membrane slice (1-14) verses integrated mass spectrometry signal intensity for six prominent peptides detected after an electro-blotting experiment.

FIG. 8B shows a table of the mass-to-charge ratio and the corresponding sequence.

FIG. 9 illustrates a table listing proteins identified by peptide mass fingerprinting of MS analysis of the peptides eluted from the different membrane slices.

FIG. 10A illustrates a top-view of an elution chamber for collection of membrane processed peptides according to the present teaching.

FIG. 10B illustrates a side-view of elution chamber for collection of membrane processed peptides according to the present teaching.

FIG. 11A illustrates a top-view of a collection plate that is designed to hold individual gel lanes.

FIG. 11B illustrates a side-view of a collection plate that is designed to hold individual gel lanes.

FIGS. 12A-D illustrate the preparation of samples for analysis in a MALDI mass spectrometer according to the present teaching.

### DESCRIPTION OF VARIOUS EMBODIMENTS

Reference in the specification to “one embodiment” or “an embodiment” means that a particular feature, structure, or characteristic described in connection with the embodiment is included in at least one embodiment of the teaching. The appearances of the phrase “in one embodiment” in various places in the specification are not necessarily all referring to the same embodiment.

It should be understood that the individual steps of the methods of the present teaching may be performed in any order and/or simultaneously as long as the teaching remains operable. Furthermore, it should be understood that the apparatus and methods of the present teaching can include any number or all of the described embodiments as long as the teaching remains operable.

The present teaching will now be described in more detail with reference to exemplary embodiments thereof as shown in the accompanying drawings. While the present teaching is described in conjunction with various embodiments and examples, it is not intended that the present teaching be limited to such embodiments. On the contrary, the present teaching encompasses various alternatives, modifications and equivalents, as will be appreciated by those of skill in the art. Those of ordinary skill in the art having access to the teaching herein will recognize additional implementations, modifications, and embodiments, as well as other fields of use, which are within the scope of the present disclosure as described herein.

Post-separation protein and peptide detection may take place by one or more of several different methodologies. Gel electrophoretic separations may be stained in a specific or non-specific manner in order to visualize the discreet post-run protein(s) locations. Once observed, individual protein bands may then be excised from the gel for further processing. Another means of removing proteinaceus material from gels, post-separation, is by electrophoretic blotting. Electrophoretic blotting also transfers samples by electrophoresis, however, it is often done in a perpendicular or orthogonal direction to that of the proceeding separation, and the samples themselves moves out of the separating gel and onto a capture membrane or substrate. Once blotted from the gel onto a capture substrate, the separated sample may again be detected by staining or used for some further study. Regardless of the method, typical gel electrophoresis and electro-blotting schemes are performed to separate protein mixtures while the detection methodology utilized depends ultimately on the type of information that is desired from the experiment.

Presently, mass spectrometry is a widely used technique for protein and peptide analysis. While several different types of mass spectrometry analyzers exist, they all rely on an accurate determination of molecular mass by measurement of mass-to-charge ratio as the means of determining the presence of an analyte(s). Two common, and often sequential, modes of mass spectrometry analyses for proteins and peptides are MS and MS/MS (tandem MS) modes. Together, these two modes of mass spectrometry are capable of determining the masses of proteins and peptides, helping to elucidate chemical structure and elemental composition.

The MS/MS mode provides amino acid sequence information, which allows for accurate protein identification. MS/MS mass spectrometry requires a mass spectrometer that performs at least two mass spectrometry steps that are typically divided by a molecular fragmentation event. For example, initial mass analysis of a sample can be used to provide a mass inventory of the analytes that are present, and then one or more of the masses identified in round one (MS) may be specifically selected for a second round of mass analysis (MS/MS). Prior to this MS/MS step of analysis, the selected analytes are induced to fragment into their constituent parts and building blocks (amino acids, and amino acid chains in the case of proteins, and peptides). The accurate detection of these building blocks in the MS/MS step of mass analysis is then used to piece together the composition and the sequence (molecular connectivity) of these building blocks. This information can then be used to accurately identify the source protein or parent protein from which the fragments derived with statistical degrees of certainty.

One prerequisite to efficient MS and MS/MS analyses for protein determination is that the material being analyzed exists within an effective operational mass range. Often, in order to fragment efficiently, or to provide useful data, proteins and peptides have to be within a workable mass range (approximately 500-5000 atomic mass units (amu)). While this mass range is approximate and not exclusive, and may vary depending on the mass spectrometer, the bounds exist because protein and peptide sequences that are too large (high mass) often do not fragment efficiently, and molecular sequences that are too short (low mass) yield limited information with regards to their usefulness for protein identification. A common means for processing source protein material to help insure that the resulting units (peptides) lie within a useful mass-range for MS and MS/MS analyses is the employment of proteases (proteolytic enzymes).

Proteases are proteins that occur in all living organisms. While proteases serve a variety of physiological functions, the net results of their operations are the breakdown of proteins by cleavage (catalysis of hydrolysis) of proteins into shorter peptide fragments. While different classes of proteases exist based on their mechanism and/or target of action, or degree of their specificity in action, several endoproteases or endopeptidases (internally cleaving) enzymes are widely and intensively used in biological mass spectrometry and biological analytics in general. Examples include trypsin, chymotrypsin, pepsin, papain, elastase, Lys-C, Glu-C, and others. The primary usefulness of these enzymes in biological mass spectrometry is that they are site-specific enzymes that cleave at particular places and in a limited manner, with the end result being the efficient production of peptides with masses compatible with MS and MS/MS analysis.

The present teaching relates to the simultaneous capture and enzymatic cleavage (digestion) of protein and peptide material so that the end result is a sample that is concentrated and processed in a manner that is directly amenable to mass spectrometry.

Two known techniques for processing proteins separated by gel electrophoresis are described in U.S. Pat. No. 6,221,262 and U.S. Patent Publication No. 2003/0175844 A1 to Nadler et al. The methods described in these patent documents use two discreet and independent substrates, one for the digestion of proteins into peptides and another for the capture of those peptides. Because these earlier methodologies use two distinctive substrates formed of synthetic polymer membranes, they are only compatible with electrophoretic sample application, since they require the application of an electrical field in order for the analytes to migrate through the enzyme containing substrate and onto the capture substrate. Furthermore, in these known methods, the second, or capture substrate, also serves as the surface of analysis in the mass spectrometer.

In contrast, methods according to the present teaching use the substrate to perform both digestion of proteins into peptides and the capture of those peptides. Additionally, the substrate according to the present teaching does not need to serve as the analytical surface for mass spectrometry. This can be advantageous because having the substrate serve as the analytical surface for mass spectrometry can compromise the performance of the mass spectrometer. Moreover, in these methods according to the present teaching, there is no time restriction for enzyme digestion. The methods are designed so that after the sample is applied, regardless of the manner of application, the substrate may be allowed to incubate as long as desired, which is highly desirable for some applications.

Thus, the present invention relates to a substrate that has dual functionality allowing for the enzymatic digestion of protein(s) and polypeptides into their constituent peptide fragments, which are then absorbed and/or otherwise retained by the membrane until they are deliberately released by the application of an appropriate elution solvent. It is important to note that in some methods according to the present teaching, a certain order of the steps is used. For example, applied proteins may first be digested into their constituent peptides followed by the capture and retention of these peptides. Alternatively, applied proteins may first be captured and retained then followed by their deliberate release and digestion. Also, in some methods according to the present teaching, depending on the capture solutions, some initial digestion may be followed prior to capture of large protein fragments, followed then by additional digestion upon transfer to a solution more favorable for digestion.

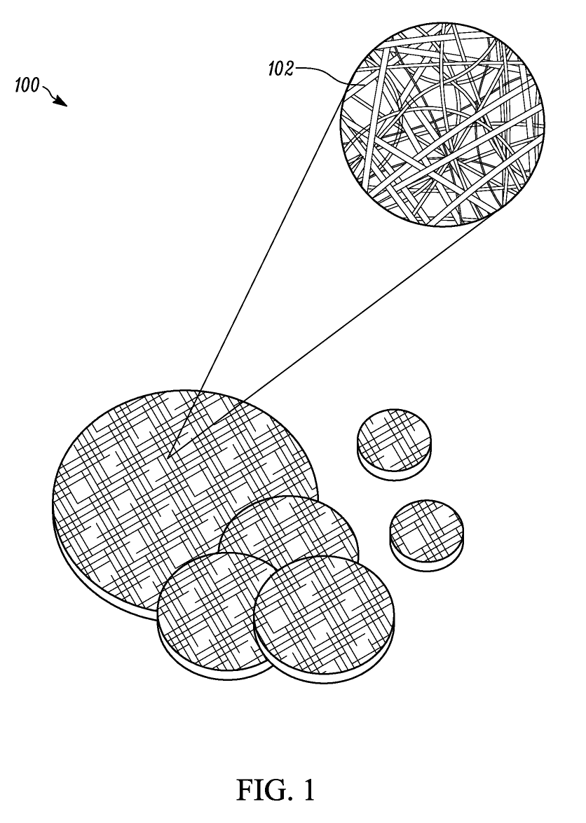

FIG. 1 illustrates substrate material 100 comprising porous glass fiber and porous membranes and discs 102 according to the present teaching. In various embodiments, the substrates of the present teaching comprise porous silica-based materials (glass) in the form of frits, discs, plates, or glass fibers that are woven or matted. These porous silica based glass material form a membrane of woven and matted glass fibers, a porous glass frit, or porous glass plate. Various silica-based substrates are readily available from commercial sources. For example, silica substrates in the form of loose fibers or fibers woven, matted, chemically cross-linked or bound together to form silica membranes and discs, are manufactured by Pall Corporation (Ann Arbor, Mich.) or Millipore Corporation (Billerica, Mass.).

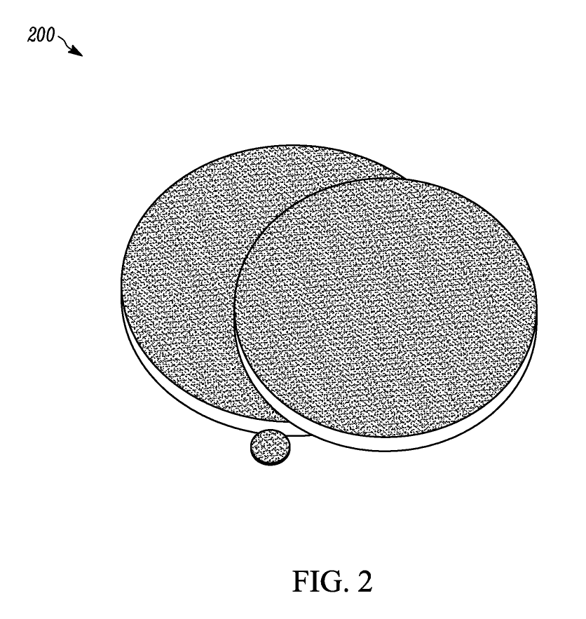

FIG. 2 illustrates sintered glass frits according to the present teaching. Fritted glass substrates 200 are manufactured from particles of glass that are fused or sintered into a solid, but porous glass body. These fitted glass substrates are commercially available from sources including Adams & Chittenden (Berkeley, Calif.) or Schott (Elmsford, N.Y.). Open-cell porous glass plates, such as VYCOR®, are commercially available from Corning Incorporated.

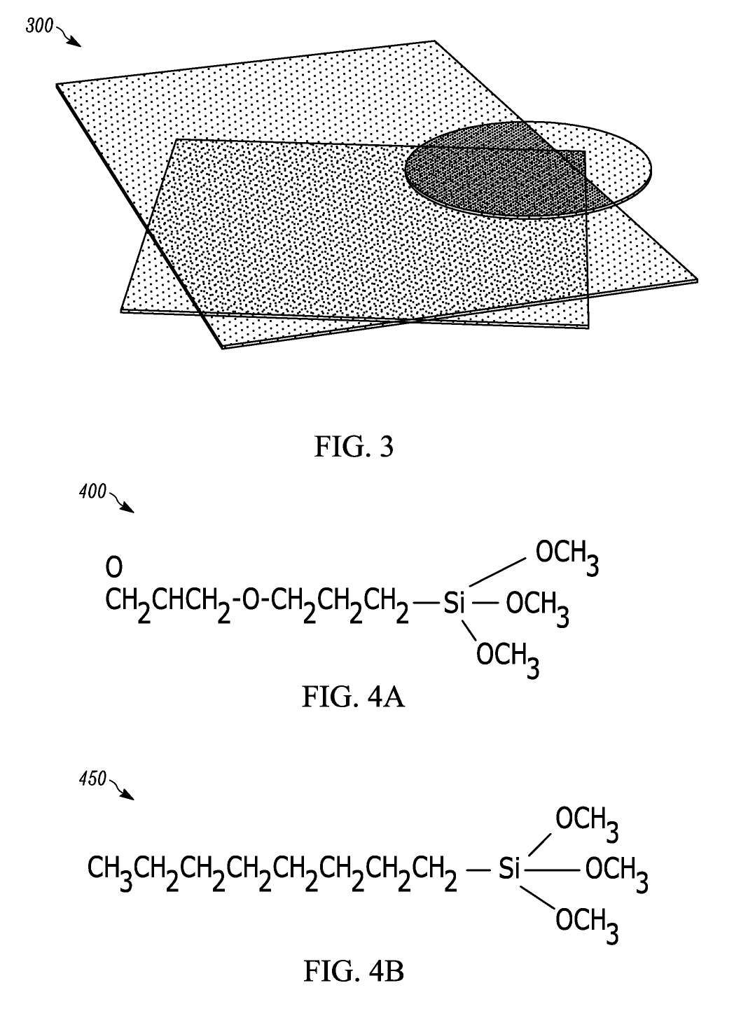

FIG. 3 illustrates an embodiment of porous glass plates 300 according to the present teaching. The outer dimensions of the glass plates 300 may be any length, width, and depth. The pores in the glass forming the glass plates 300 may be amorphous and can be of any size. There is no requirement for the pores to be in an orderly array or perforated pattern of any sort. The pores may be amorphous or of any size and shape and do not have to be uniformly spaced in an orderly array and do not need to conform to any perforated pattern. Thus, in various embodiments, there are numerous configurations for the pores contained within the membranes, frits or plates and the outer dimensions of the substrate, and the configurations may be crafted to any dimension to best suite a particular application.

The porous glass substrates according to the present teaching are modified to perform two specific functions. First, the porous glass substrates serve as a capture substrate, where analyte(s) of interest are captured by chemical adsorption via hydrophobic and/or electrostatic interaction within the modified silica substrate. Second, the porous glass substrates serve as a site of biological processing by incorporating a biologically active enzyme into to the silica substrate. The adsorption and biological processing (enzymatic digestion) of the analyte(s) of interest will take place within the same modified porous glass substrate. The processed material are eluted or released for further analysis after the desired incubation time is past.

The bound, or incorporated, enzyme may be of any class or type, either naturally occurring or synthetic. The enzyme operates by cleaving proteinaceous material via a hydrolysis reaction into small fragments in a site-specific manner. The proteinaceus material applied to the substrates can be derive from any source and may have undergone any or no prior sample handling or modification. Furthermore, the proteinaceus material may be applied to the substrate in any manner or from any medium and may be in the form of a solid, liquid or gas. In various embodiments, one or more enzyme(s) may be coupled to any given glass substrate.

In various embodiments of the method of the present teaching, the substrate can be manipulated by one or more means to enable the bound, or captured, analyte(s) to be biologically processed to suit the particular requirements of the method and the particular properties of the bound analyte(s). The glass substrate can be manipulated by using silane chemistry to covalently link the protein and peptide capture chemistry as well as to covalently link active protease enzyme(s) on the same contiguous or adjoined substrate structure. Also, a temperature of the substrate can be increased or decreased to enhance the biological processing of the bound analyte(s). Also, the substrate can be exposed to RF energy, such as microwave energy, to enhance the biological processing of the bound analyte(s). Also, the substrate can be soaked or rinsed in a liquid to enhance the biological processing of the bound analyte(s).

One aspect of the present teaching is that the adsorptive qualities of the substrate used to manipulate the proteins and peptides remain bonded to material until such time as the proper elution conditions are specifically applied. Such adsorptive qualities have been deemed undesirable in the enzyme substrates described in the known methodologies. Thus, various embodiments of the present teaching relate to membranes and substrates comprising porous glass that is modified to capture proteins and/or polypeptides, and to enzymatically digest them into their constituent peptides and retain the digested material within that same substrate until their deliberate release for detection, identification or further processing.

In one method according to the present teaching, samples are applied to the substrate by various means, including mechanical placement, chromatographic effluent, or electro-blotting. For example, samples may be applied to the substrate by gas, aerosol, liquid, liquid suspension, or solid application, such as applying a piece of biological tissue. The applied sample may have undergone any or no prior processing depending upon the specific application. Protein/peptide adsorption chemistry is then performed by either hydrophobic, electrostatic, or affinity based modes of chemical interaction to bind or capture the sample. It should be understood that more than one type of adsorption chemistry can be used simultaneously on any given glass substrate.

Sample digestion is then performed. The sample bound within the substrate may undergo further treatment to expedite, control, or to enhance the digestion process during post processing. For example, during post processing, the substrate can be heated or cooled to various temperatures, irradiated by RF radiation (such as microwave radiation), soaked, rinsed, and/or dried.

Samples are released from the substrate by various means of elution with the proper elution solvent. For example, if the mode of analyte adsorption is one of hydrophobic interaction, the application of a solution suitably high in organic solvent composition will be used to release the bound analyte. Similarly, if the mode of analyte adsorption is one of electrostatic interaction (charge-charge), the application of a solution suitably high in ionic content (e.g. salt) will be used to disrupt the adsorption and release the analyte. Similarly, if adsorption is of an affinity type of interaction (lock-and-key), the application of a solution suitably high in composition with a competitively interacting compound(s) will be used to release the analyte of interest. Analytes of interest bound to the substrate may be released in unison or in any sequential manner.

Collection of bound analytes may be performed in numerous ways. Eluted sample may be collected onto another surface or substrate, and may be collected in wells or enclosures of any size or shape. The concentrated, digested proteinaceus samples resulting from the methods of the present teaching can be used for any further processing.

One aspect of the present teaching is that an elution chamber with collection plates of specific construction can be used to enable the elution of large sections of membrane into wells of appropriate size so that the resolution of the separation is maintained. FIGS. 4A and 4B illustrates chemical diagrams for 3-glycidoxypropyl trimethoxysilane 400 and for n-octadecyltrimethoxysilane 450, which are two different and commonly available silane-compounds that are used for surface modification according to the present teaching. The silicone atom (Si) in each molecule is connected to four pendant arms, three identical (methoxy —OCH3), and one different 3-glycidoxypropyl in FIG. 4A and n-octadecyl in FIG. 4B. The epoxy functionality at the terminal end of the molecule in FIG. 4A can be readily induced to react with amines located on the N-terminus and lysine moieties in all proteins, including proteases. The n-octadecyl functionality on the molecule of FIG. 4B is a hydrophobic moiety that is commonly used as a reversed phase chromatographic resin for adsorption of proteins and peptides by hydrophobic interaction. The three methoxy pendants on both molecules may be induced to react with one another, as well as with hydroxyl functionalities present on silica surfaces, to form a stable covalent bond to the surface of the glass.

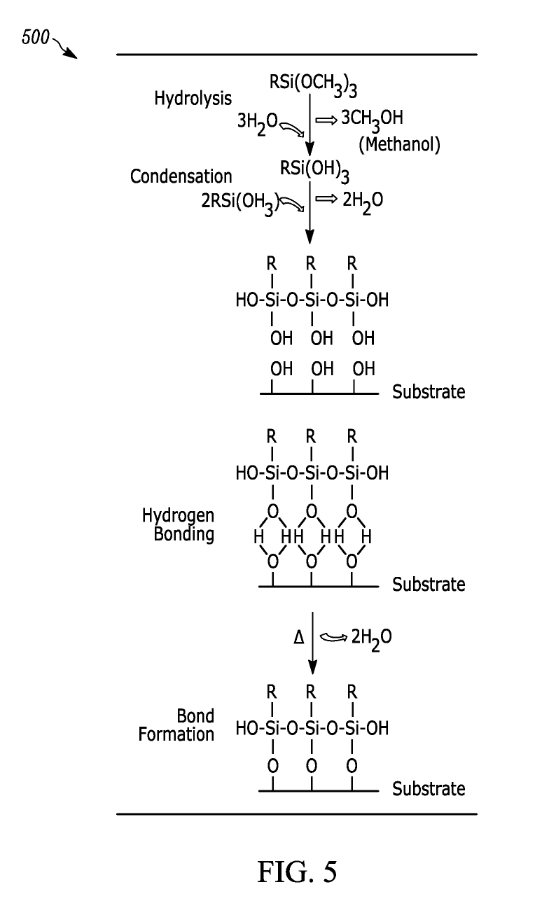

FIG. 5 illustrates a reaction mechanism 500 wherein the three methoxy pendants on both molecules in FIGS. 4A and 4B may be induced to react with one another, as well as with hydroxyl functionalities present on silica surfaces, to form a stable covalent bond to the surface of the glass for surface modification. One skilled in the art will appreciate that the silane-chemistry is well known and has been extensively studied, and the figures shown herein are only meant to outline the basic mechanism of attachment of silanes to a silica substrate.

Thus, one aspect of the present teaching is using common silica substrates and enabling them with different chemistries, such as applying silanes, and then using these modified substrates to perform tasks in new processes. There are numerous different silanes that may be used to attach proteases to silica. There are also numerous different silanes that may be used to adsorb proteins and peptides by multiple modes of chemical interaction. Many of the various schemes envisioned for the performance of these tasks will be viable and productive.

There are various methods of applying silanes to silica substrates according to the present teaching. In one method according to the present teaching, glass substrates are cleaned and prepared prior to silanization. The protocol involves rinsing in distilled water or a diluted acid solution (e.g. 5% HCl) to remove surface impurities. Additionally, substrates are treated with a strongly acidic and highly oxidizing solution called piranha solution (1:1 mixture of 50% aqueous sulfuric acid: 30% hydrogen peroxide). Piranha solution is utilized for cleaning as well as for its ability to increase the hydroxyl content (—OH) on the silica substrate surface. Depending on the particular silane-compound being applied (solubility, chemical reactivity), application can be made with either aqueous of organic solution, or some combination thereof. Additionally, deposition may take place by immersion of the substrate into the silane-containing solution, or the solution may be applied to the substrate by means of vapor, spray or spin-on application, as outlined in common protocols.

A protocol according to the present teaching that has been used successfully includes first forming a 7×7 inch sheet of Type A/C glass fiber (Pall Corporation, Ann Arbor, Mich.). Rinsing the sheet for 10 minutes in 200 uL of deionized water and then immersing the sheet to into 100 uL of piranha solution (1:1 mixture of 50% aqueous sulfuric acid: 30% hydrogen peroxide). The sheet is then rinsed three times in deionized water and allowed to dry.

Once dried, the membrane is placed into an 8×8 inch glass dish containing 150 uL silanizing solution. Silanizing is formulated using a volume/volume composition of 95% ethanol, 2% DI water, 1% actetic acid, 1% 3-glycidoxypropyl triethoxysilane, and 1% n-octadecyltriethoxysilane. This silane formulation is allowed to stand for 5 minutes prior to the addition to the glass membrane. After immersing the membrane, the dish is covered and placed on an oscillation table rotating at about 50 rpm for about five minutes. The sheet is then removed from the silane solution and rinsed briefly by immersion in 150 μL 95% ethanol, 5% DI H₂O.

Post rinse, the membrane is suspended in air from a metal rod using a metal clip, and then allowed to dry at room temperature for approximately two hours. Once dried, the membrane, which is suspended in air from a metal rod using metal clip, is place in an oven preheated to 85 degrees C., and allowed to cure for 10 minutes. Post curing, the now silanized membrane is added to 100 μL of 50 mM Na₂CO₃, 50 mg trypsin, 100 mg benzamidine hydrochloride, in 20% ethanol (v/v), and again covered and placed on an oscillation table rotating at about 50 rpm for approximately eight hours. Post incubation in enzyme, the membrane is rinsed briefly in 50% ethanol to remove residual unbound enzyme and benzamidine, and then rinsed in DI H₂O, placed in a zip-lock plastic bag, and placed into a refrigerator at about four degrees C. until further use.

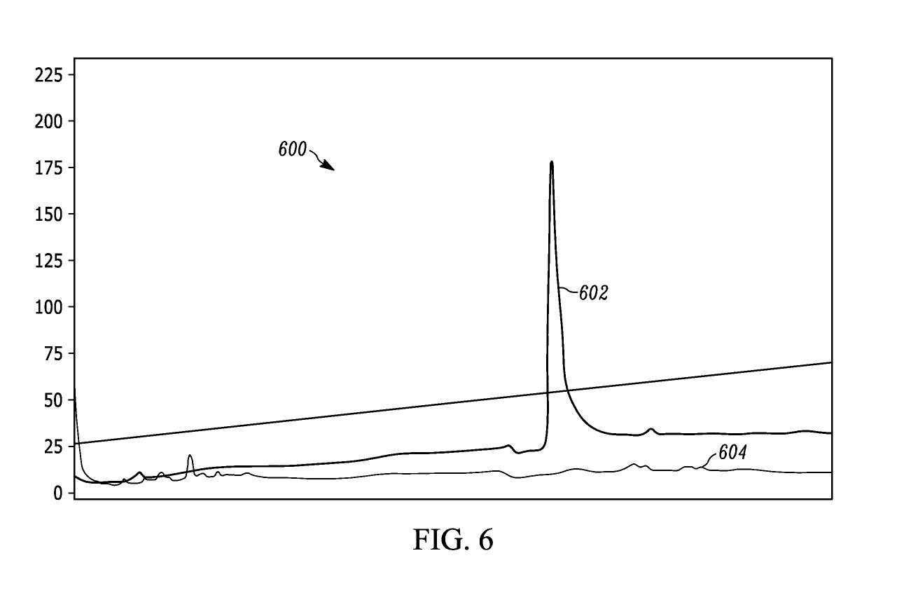

FIG. 6 illustrates LC chromatograms 600 of a Bovine Serum Albumin after the addition of a 5 mm² piece of capture/digestion membrane according to the present teaching. A LC chromatogram 602 is presented for a 10 μL injection of a Bovine Serum Albumin sample (0.5 mg/mL, 200 uL) before the addition of 5 mm² piece of capture/digestion membrane. A LC chromatogram 604 is presented for a 10 μL injection of a Bovine Serum Albumin sample (0.5 mg/mL, 200 uL) after the addition of 5 mm² piece of capture/digestion membrane and an eight hour incubation period.

Thus, FIG. 6 shows an overlay of two LC chromatograms of the same 0.5 mg/mL protein sample before 602 and after 604 the application of the capture and digestion membrane. As shown in FIG. 6, before the addition of the membrane, a clear and distinct protein peak appears in the chromatogram. After adding the sample to the membrane, it was allowed to incubate for an eight hour period before it was removed. The material bound by the membrane was removed by applying a solvent that was 75% acetonitrile and 25% water. The LC chromatogram 604 performed after the addition of the capture/digestion membrane, indicates that peptides are seen while the protein peak is missing. Analysis of the sample shows the protein peak is gone with the appearance of several peaks having reduced mass that correspond to peptides (smaller pieces or sections of the parent protein). Analysis of these masses shows that they are peptides resulting from the tryptic digestion of Bovine Serum Albumin (the parent protein). Not only was the Bovine Serum Albumin protein digested, but the peptides were retained within the membrane until released by the appropriate mechanism (addition of an organic solvent, in this case).

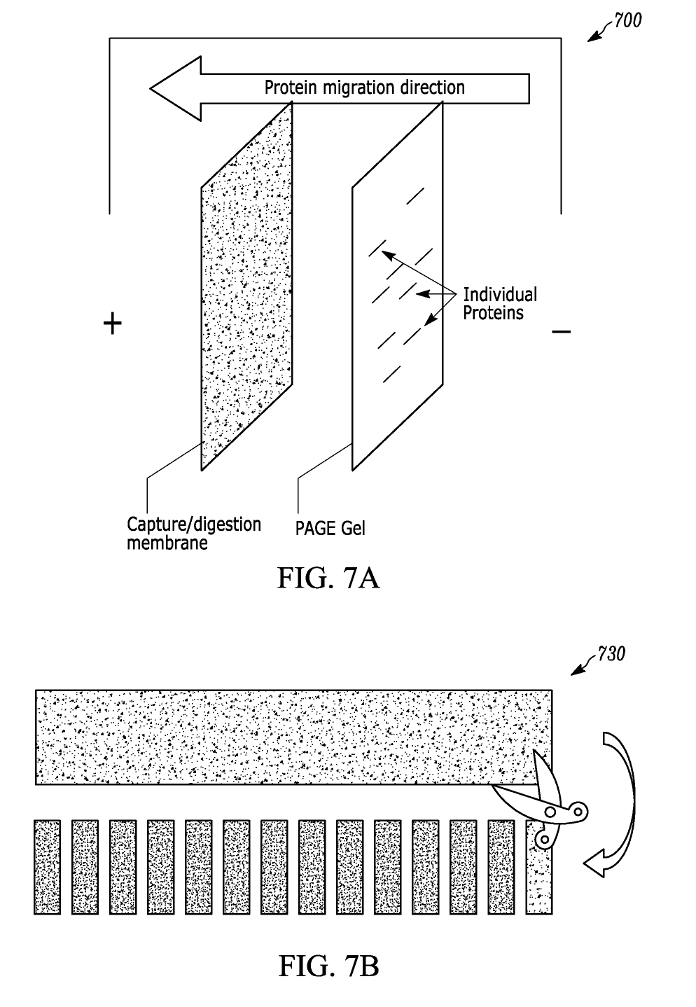

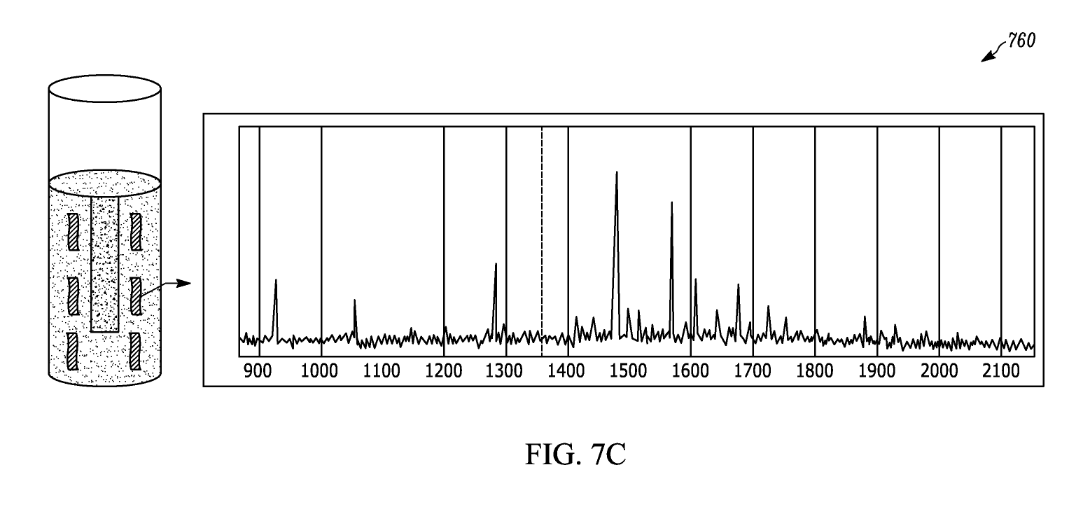

FIG. 7A illustrates a schematic of an electro-blotting procedure 700 according to the present teaching. With this electro-blotting procedure 700, proteins are separated by sodium dodecyl sulfate (SDS) polyacrylamide gel electrophoresis (PAGE). Sodium dodecyl sulfate is a detergent that dissociates and unfolds oligomeric proteins into its subunits. Such a separation technique is useful for molecular weight analysis of proteins.

The gel is incubated in an appropriate blot buffer. Either the entire gel or any section thereof is then placed in an electric field and the separated proteins that reside in that gel are induced to migrate out of the gel. Placing the capture/digestion membrane in the path of the proteins will allow for their capture and digestion.

FIG. 7B illustrates a resulting post blot membrane 730 that can be cut to preserve separation. In the post blot step, the membrane may be treated in any manner deemed suitable to the goals of the experiment.

FIG. 7C illustrates a mass spectrum 760 of slices that are eluted to release peptides for analysis or further processing. In the example presented, the membrane is sliced into sections in order to preserve the protein resolution of the PAGE-Gel separation upon release of the peptides. After slicing, the individual membrane pieces are eluted and analyzed. In some methods according to the present teaching the peptides may be subjected to further treatment.

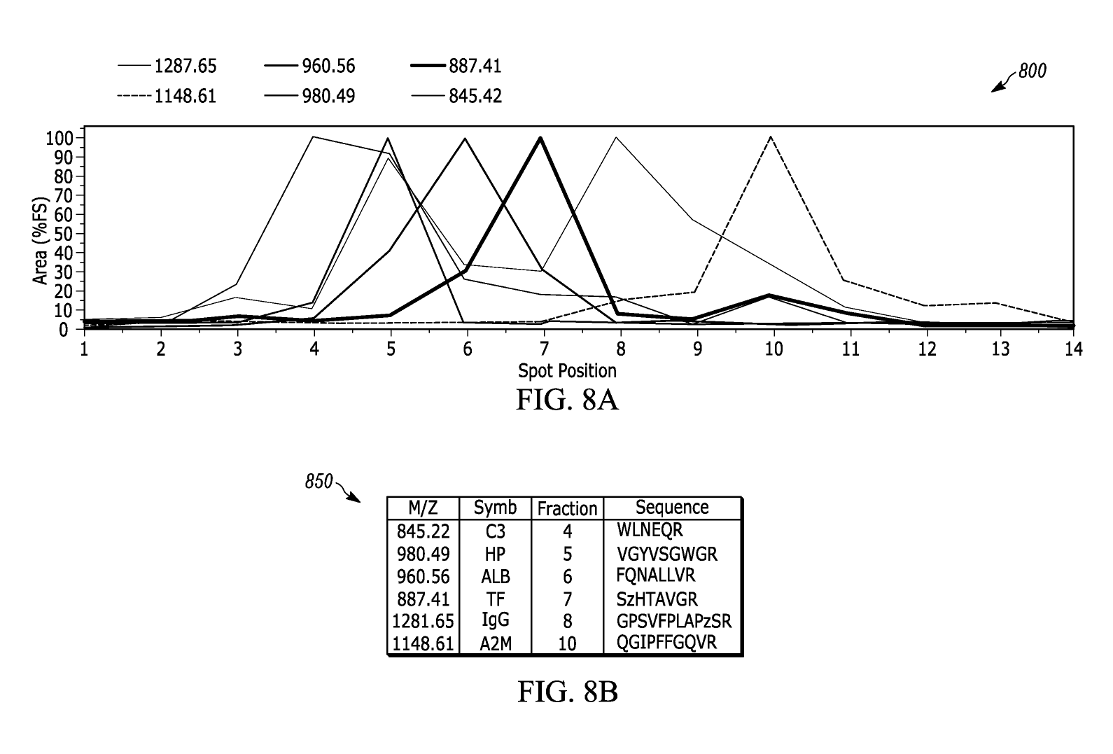

FIG. 8A illustrates a plot 800 of membrane slice (1-14) versus integrated mass spectrometry signal intensity for six prominent peptides detected after an electro-blotting experiment. FIG. 8B shows a table 850 of the mass-to-charge ratio and the corresponding sequence. As shown in FIG. 8A, signal intensity for each of the peptides tends to localize in a given membrane slice. This localization indicates the separation position (polyacrylamide gel electrophoresis) of the protein to which the peptide belongs. The protein was blotted out of the gel and into the capture membrane where it was digested into its constituent peptides.

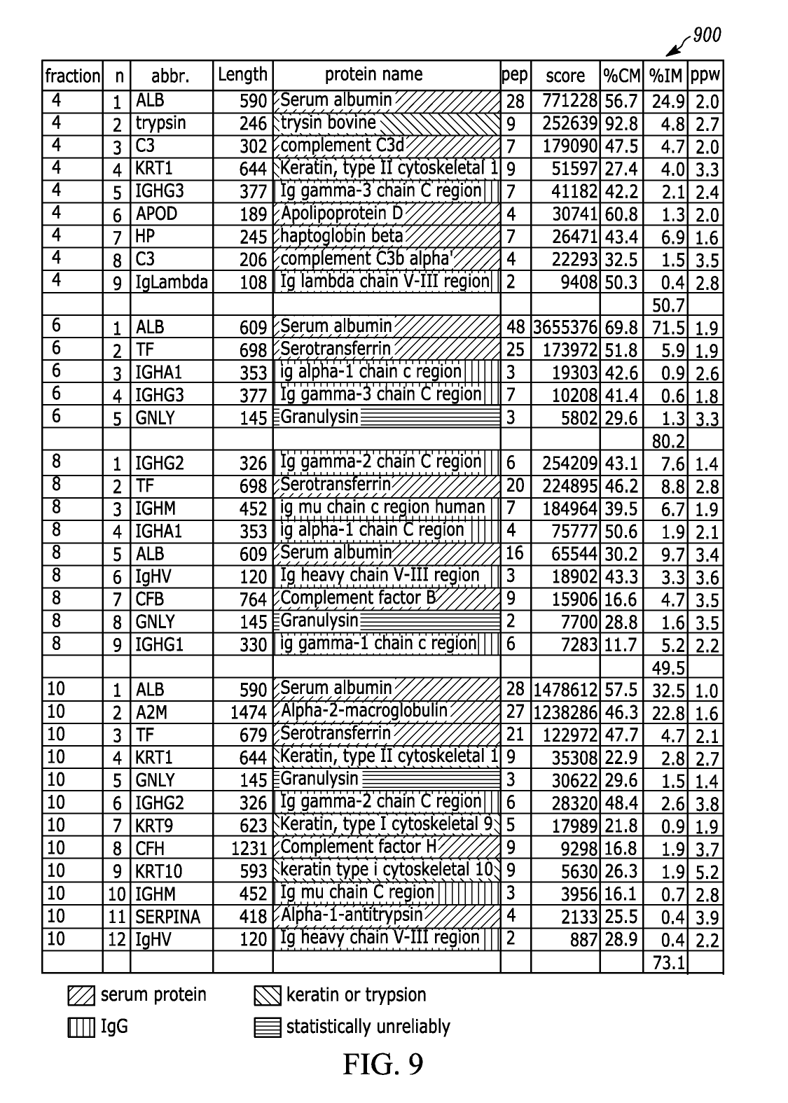

FIG. 9 illustrates a table 900 listing proteins identified by peptide mass fingerprinting of MS analysis of the peptides eluted for the different membrane slices. The ability of the membrane to bind peptides and keep them spatially arranged has several advantages. One advantage is that by having the peptides that derive from a given localized protein helps combat the problem of highly abundant proteins dominating the detection space. Another advantage of having peptides derived from a given protein localized is that it better enables protein identification by peptide mass fingerprinting. Yet another advantage is that it is possible to define the parent protein(s) by peptide mass fingerprinting with the knowledge of the cleavage rules for the particular enzyme used, and by having accurate mass measurements of multiple peptides derived from each protein. Peptide mass fingerprinting is a powerful and effective protein identification technique capable of identifying multiple proteins from a single MS spectrum. Peptide mass fingerprinting can be used exclusively or in conjunction with subsequent peptide chromatographic separation and MS/MS analysis to deliver further proteome characterization.

The table in FIG. 9 shows several proteins as being identified in each fraction. Each protein is given a “score” to indicate the confidence or relative certainty of that identification. Generally, proteins that score above a certain threshold value are considered to be reliably identified. For the proteins displayed in the table of FIG. 9, those with a score >10,000 are considered statistically reliable and those with a score <10,000 are considered statistically unreliable. Abundant serum proteins, such as albumin and transferrin, are detected across multiple fractions. The results show that less abundant proteins are localized to one or two of the membrane slices.

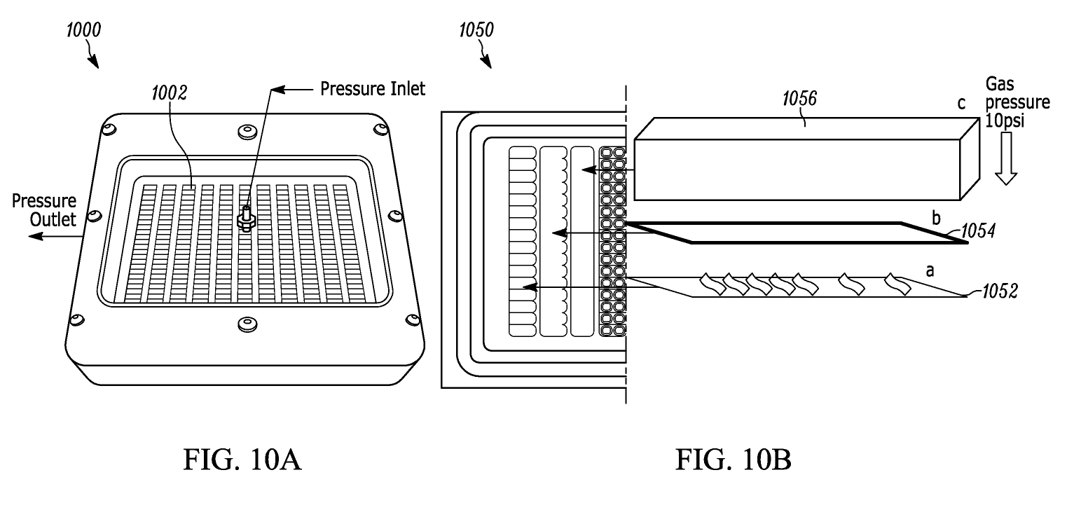

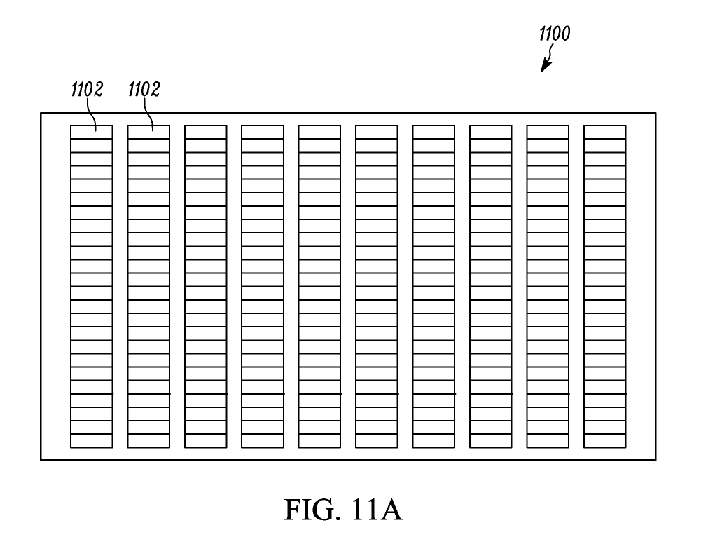

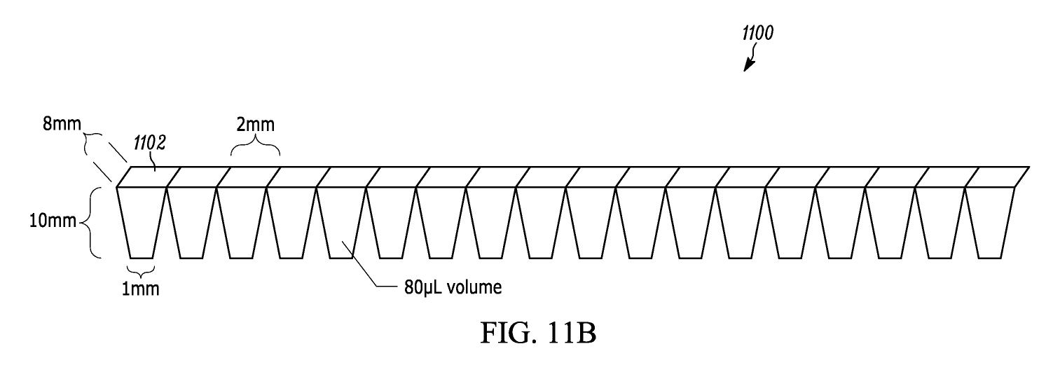

An elution chamber and the design of two prototype sample collection plates, are presented in FIGS. 10A, 10B, 11A and 11B for the automated elution of samples from the digestion/capture membrane of the present teaching. The elution chamber will cause the elution of bound analyte by using positive gas pressure, such as air or an inert gas like N₂ or argon. A pressure inlet on the face of the chamber will allow gas of regulated pressure to enter the chamber. This positive gas pressure will apply a constant force to elution solvent placed on top of the membrane and cause this solvent to flow into and through the membrane. The volume of elution solvent is controlled so that enough solvent passes through the membrane to achieve an efficient elution. The solvent volume is regulated by having it added to a sponge. This sponge rests onto of the membrane and helps to insure even elution solvent distribution. Upon application of air pressure, the elution solvent flows out of the sponge and into and eventually through the membrane. After passing through the membrane, the elution solvent is collected in a collection plate set underneath it.

FIG. 10A illustrates a top-view of an elution chamber 1000 for collection of membrane processed peptides according to the present teaching. The elution chamber 1000 includes a modified micro-titer plate 1002 machined to hold membrane strips dimensioned to fit in individual gel lanes. The membrane strips containing peptides are covered with filter paper. An inert sponge holds an appropriate volume of elution solution. The filter paper and inert sponge are required to help regulate and evenly distribute the elution solvent on top of the membrane.

FIG. 10B illustrates a side-view of the elution chamber 1000 for collection of membrane processed peptides according to the present teaching. The side-view shows the membrane 1052 containing peptides overlayed with filter paper 1054 and a porous sponge 1056 that are positioned on top of the membrane 1052. The filter paper 1054 and porous sponge 1056 are used to control elution volume and to assure uniform distribution of the elution solvent across the membrane surface.

FIG. 11A illustrates a top-view of a collection plate 1100 that is designed to hold individual gel lanes in collection wells 1102. The collection plate includes approximately 2 mm sized collection slots that approximate 2× the size of a well defined protein band as visualized by coomassie staining Multiple analyses of complex protein samples have demonstrated that fractions of this size lead to approximately 75% of the identified proteins residing in a single fraction. This sized fraction is deemed a good starting point for prototype elution chamber construction. It should be understood that the collection wells can be adjusted to any size as necessary to best fit the resolution of a gel or the objectives of the experiment.

FIG. 11B illustrates a side-view of a collection plate 1100 that is designed to hold individual gel lanes. The dimensions of the wells 1102 for one particular collection plate are shown in FIG. 11B. The sample collection plate as described herein can be used for general sample collection. Once the elution solvent has passed through the membrane and eluted, the bound analyte resides in the collection well that is located directly below its original location on the membrane. The analyte resides in this elution solvent and may be used for any purpose. As shown in FIG. 11B, each well 1102 is approximately 2 mm wide, 8 mm long and 10 mm deep and has a volume of 80 microliters. Some applications employ wells 1102 with the same dimension as the enclosed wells of the standard collection plate, but for these applications, the wells are open at the bottom.

One aspect of the present teaching is the creation of a specialized collection plate constructed to allow direct interface with a mass spectrometry designed for promoting sample ionization by matrix assisted laser desorption ionization (MALDI). The MALDI mass spectrometry technique uses what is known as a “matrix” substance to help promote the gas phase ionization of analyte(s). There are many commonly used MALDI matrices, but most of them can be classified as small organic acids. Matrices are usually applied dissolved as liquid solution. After application, the carrier solvent evaporates and the remaining matrix molecules form into solid crystals. These matrix crystals then function to absorb energy provided by a laser causing thermal excitation and the rapid expansion into the gas phase. During the process of matrix excitation and gas phase expansion, species, sample, compounds, materials etc. in contact with the matrix may also be collaterally induced into the gas phase. During this process, these comingled sample molecules may also be induced to ionize and rendered controllable and detectable by the mass spectrometer.

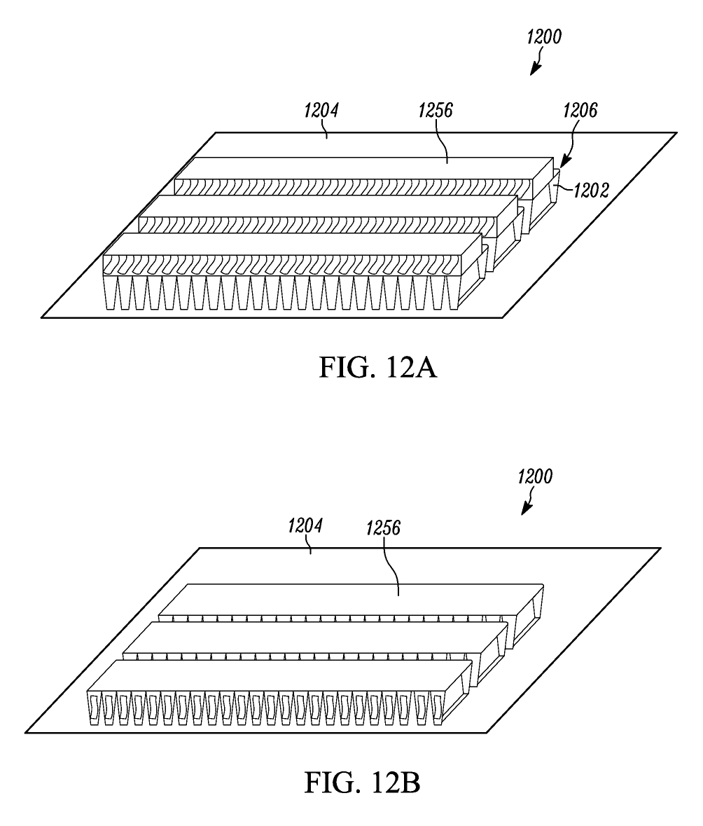

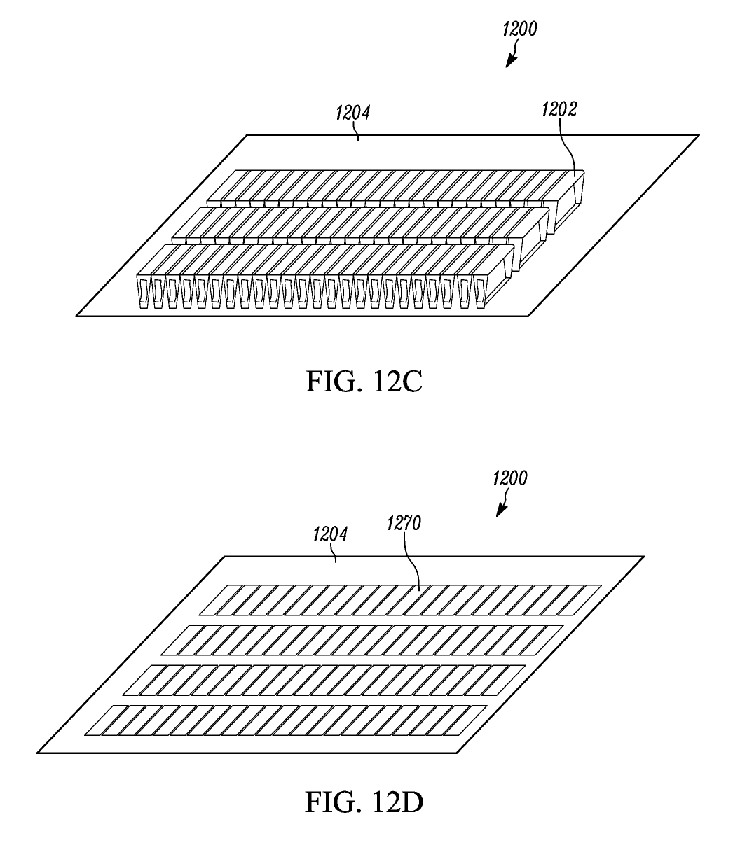

FIGS. 12A-D illustrates the preparation of samples for analysis in a MALDI mass spectrometer according to the present teaching. A specialized interface plate can be constructed for an elution chamber according to the present teaching. The dimensions of the elution chamber 1200 for a specialized MALDI interface plate are similar to the dimensions of standard collection plates with segmented collection wells that are used for general sample collection. However, in the specialized elution chamber, the wells 1202 are open at the bottom and the structure forming the sides of the wells is only affixed to a plate 1204 that completes the well. For this specialized MALDI interface plate 1200, the collection wells 1202 are affixed on top of a plate 1204 that is detachable and composed of a material that is electrically conductive so that it may be used directly as one of the electrodes in the applied accelerating field in a MALDI mass spectrometer. That is, the bottom of the collection well is effectively the MALDI target 1204 as shown in FIG. 12A. Therefore, the elution chamber operates in a manner similar to the elution chamber designed for general sample collection. However, the contact surface at the bottom of the wells is detachable.

The elution chamber 1200 is configured to process eluted sample material so that it can be directly interfaced with a MALDI mass spectrometer for sample interrogation. For example, a membrane 1206 that operates by capturing peptides by a reversed-phase mode is eluted by application of an organic solvent (hydrophobic in nature, a solvent less polar than water). Typical MALDI matrix formulations contain between 50-75% organic solvent. Therefore, the MALDI matrix itself may be used for membrane elution. The matrix solution is then allowed to dry, or expedited to dry, within the wells and directly on the well bottom. After the sample has been allowed to dry, the bottom surface 1204 is detached and placed into the MALDI mass spectrometer for sample analysis.

The template defining the wells 1202 can then be removed and the remaining target analyzed. In this manner, this sample collection plate 1204 serves as a ready interface to sample detection by MALDI mass spectrometer and also serves to concentrate the sample.

FIG. 12A illustrates an elution chamber for interfacing to MALDI wherein the inert sponge 1256 containing elution solvent is placed over the membrane 1206. The membrane 1206 sits on top of the sample collection wells 1202, in a manner identical to that described herein for the standard elution plate. The only difference is that collection wells 1202 are open bottomed wells. The open bottom wells are fixed to the MALDI target 1204.

FIG. 12B illustrates the elution chamber 1200 after positive gas pressure has been applied forcing the elution solvent out of the inert sponge 1256, through the membrane 1206, and into the collection wells 1202. For this MALDI application, this elution process can use the MALDI matrix solution as the eluent solution. Pressure is applied at the top surface to elute the sample material. The eluted sample material is collected in the wells below. The wells contain the eluted sample material because the collection plate is affixed to a suitable MALDI target 1204 that acts to form the bottom of the well that contains the elution solution.

FIG. 12C illustrates the elution chamber for interfacing to MALDI 1200 with the membrane 1206 removed allowing matrix solution containing eluted peptides to dry and concentrate on the target surface 1204.

FIG. 12D illustrates the MALDI target surface 1204 after the removal of the collection well plate 1202. The resulting MALDI target contains the dried matrix with the anlayte(s) comprising materials processed by the digestion/capture membrane deposited on regions 1270 corresponding to the bottom of collection wells in collection well plate 1202. This resulting MALDI target can then be placed directly into the MALDI mass spectrometer for sample analysis.

### EQUIVALENTS

While the applicant's teaching are described in conjunction with various embodiments, it is not intended that the applicant's teaching be limited to such embodiments. On the contrary, the applicant's teaching encompasses various alternatives, modifications, and equivalents, as will be appreciated by those of skill in the art, which may be made therein without departing from the spirit and scope of the teaching.

What is claimed is:

1. A mass spectrometry substrate for performing adsorption and biological processing, the mass spectrometry substrate comprising:
a) a porous silica-based material comprising a chemical adsorption material that captures analyte(s) of interest; and
b) a biologically active material incorporated into the porous silica-based material, the biologically active material performing biological processing on the analyte(s) of interest.

2. The mass spectrometry substrate of claim 1 wherein the biologically active material comprises an enzyme and the biological processing comprises enzymatic digestion.

3. The mass spectrometry substrate of claim 2 wherein the enzymatic digestion comprises enzymatic digestion of protein(s) and polypeptides into their constituent peptide fragments.

4. The mass spectrometry substrate of claim 2 wherein the enzymatic digestion is accomplished by immobilization of a naturally occurring enzyme.

5. The mass spectrometry substrate of claim 2 wherein the enzymatic digestion is accomplished by immobilization of a synthetic or artificial enzyme.

6. The mass spectrometry substrate of claim 1 wherein the analyte(s) of interest are captured by hydrophobic interaction.

7. The mass spectrometry substrate of claim 1 wherein the analyte(s) of interest are captured by electrostatic interaction.

8. The mass spectrometry substrate of claim 1 wherein the chemical adsorption material causes a chemical interaction that is based on differential charge between the analyte(s) and the mass spectrometry substrate.

9. The mass spectrometry substrate of claim 1 wherein the chemical adsorption material causes a chemical interaction that is based on hydrophobic interactions between the analyte(s) and the mass spectrometry substrate.

10. The mass spectrometry substrate of claim 1 wherein the chemical adsorption material causes chemical interactions that are based on both hydrophobic interactions and on differential charge interactions between the analyte(s) and the mass spectrometry substrate.

11. The mass spectrometry substrate of claim 1 wherein the porous silica-based material comprises at least one glass frit.

12. The mass spectrometry substrate of claim 11 wherein the at least one glass frit comprises at least one sintered glass frit.

13. The mass spectrometry substrate of claim 1 wherein the porous silica-based material comprises a glass disc.

14. The mass spectrometry substrate of claim 1 wherein the porous silica-based material comprises a glass plate.

15. The mass spectrometry substrate of claim 1 wherein the porous silica-based material comprises woven glass fibers.

16. The mass spectrometry substrate of claim 1 wherein the porous silica-based material comprises matted glass fibers.

17. A method of preparing samples for mass spectrometry, the method comprising:
a) applying sample material to a porous silica-based substrate;
b) capturing analyte(s) of interest in the porous silica-based substrate by chemically adsorbing the analyte(s) of interest within the porous silica-based substrate material;
c) biological processing the analyte(s) of interest captured in the porous silica-based substrate material with a biologically active material; and
d) releasing the biologically processed analyte(s) of interest for detection and analysis.

18. The method of claim 17 further comprising cooling the substrate to enhance biological processing of the analyte(s) of interest captured in the substrate.

19. The method of claim 17 further comprising heating the substrate to enhance biological processing of the analyte(s) of interest captured in the substrate.

20. The method of claim 17 further comprising exposing the substrate to RF energy to enhance biological processing of the analyte(s) of interested captured in the substrate.

21. The method of claim 17 further comprising rinsing the substrate in a fluid to enhance biological processing of the analyte(s) of interested captured in the substrate.

22. The method of claim 17 further comprising releasing the biologically processed analyte(s) of interest for detection and analysis after a predetermined incubation time is past.

23. The method of claim 17 wherein the applying the sample material to the substrate comprises mechanical placement of the sample material on the substrate.

24. The method of claim 17 wherein the applying the sample material to the substrate comprises chromatographic effluent.

25. The method of claim 17 wherein the applying the sample material to the substrate comprises electro-blotting.

26. The method of claim 17 wherein the applying the sample material to the substrate comprises exposing the substrate to a gas or aerosol.

27. The method of claim 17 wherein the applying the sample material to the substrate comprises exposing the substrate to a liquid or liquid suspension.

28. The method of claim 17 wherein the applying the sample material to the substrate comprises placing the substrate in contact with biological tissue.

29. The method of claim 17 further comprising modifying the porous silica-based substrate by exposing the substrate to silane chemistry that covalently links proteins and peptides.

30. The method of claim 17 wherein the biological processing comprises enzymatic digestion

31. The method of claim 17 wherein the chemically adsorbing the analyte(s) of interest causes a chemical interaction that is based on differential charge between the analyte(s) and the mass spectrometry substrate.

32. The method of claim 17 wherein the chemically adsorbing the analyte(s) of interest causes a chemical interaction that is based on hydrophobic interactions between the analyte(s) and the mass spectrometry substrate.

33. The method of claim 17 wherein the chemically adsorbing the analyte(s) of interest causes chemical interactions that are based on both hydrophobic interactions and on differential charge interactions between the analyte(s) and the mass spectrometry substrate.
